(19) United States
(12) Patent Application Publication
(10) Pub. No.: US 2003/0167506 A1
(43) Pub. Date: Sep. 4, 2003

(54) Expansin protein and polynucleotides and methods of use

(75) Inventors: Dilbag S. Multani, Urbandale, IA (US); Gurmukh S. Johal, Urbandale, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc.

(21) Appl. No.: 10/102,349

(22) Filed: Mar. 20, 2002

Related U.S. Application Data
(60) Provisional application No. 60/324,182, filed on Sep. 21, 2001.
(60) Provisional application No. 60/277,847, filed on Mar. 22, 2001.

(51) Int. Cl. A01H 5/00; C12Q 1/68; C07H 21/04; C12N 9/24

(52) U.S. Cl. 800/278; 800/320.1; 435/6; 435/69.1; 435/200; 435/320.1; 536/23.2; 435/410

(57) ABSTRACT

Methods and compositions for modulating plant growth, strength and flexibility are provided. Nucleotide sequences encoding maize expansin proteins are provided. The sequence can be used in expression cassettes for modulating growth, stalk strength and flexibility. Transformed plants, plant cells, tissues, and seed are also provided. Methods for rapidly identifying and isolating a Mu-tagged recessive gene mutation in a F1 generation plant, and identification and isolation of its associated wild-type gene are also provided.

### CROSS-REFERENCE TO RELATED APPLICATIONS

This application claims the benefit of and incorporates by reference U.S. Provisional Application Ser. Nos: 60/277,847 filed Mar. 22, 2001, and 60/324,182 filed Sep. 21, 2001.

### FIELD OF THE INVENTION

The invention relates to the field of the genetic manipulation of plants, particularly the modulation of gene activity and development in plants of genes related to plant strength and flexibility.

### BACKGROUND OF THE INVENTION

Expansins are novel cell-wall-loosening proteins unique to the land plant lineage (Cosgrove, D. 2000. Nature, 407: 321-326). They were discovered on the basis of their ability to induce cell wall extension in vitro (McQueen-Mason, et al. 1992. Planta, 190: 327-331). Available evidence suggests that expansins may be the primary protein mediators of ‘acid growth’—which refers to an increase in growth rate that occurs when plant cells are placed in acidic solutions. The exact mechanism by which expansins facilitate cell wall extension remains obscure. One hypothesis holds that they destabilize non-covalent linkages, such as hydrogen bonds, between hemicellulosic wall components and cellulose microfibrils, thereby allowing turgor driven extension (creep) of plant cell wall polymers (Cosgrove, D. 2000. Nature, 407: 321-326).

Since the discovery of cucumber expansins in 1992 (McQueen-Mason, et al. 1992. Planta 190: 327-331), many homologous sequences have been cloned or identified in plant databases. Expansins now make up a large superfamily that has been divided into two broad classes, α- and β-expansins, based on sequence homology (Cosgrove, D. 2000. Nature, 407: 321-326). α-expansins, of which there appear to be 24 in Arabidopsis, exhibit a high degree of homology (more than 75% identical at the protein level) to the original expansin gene. The other class, β-expansins, is relatively more divergent than the α-expansin class. Although it shares only 20% amino acid identity with α-expansins, the two appear to have a similar structural organization. The founding member of the β-expansin class was a group-1 pollen allergen from maize, Zm1. It does exhibit a cell-wall loosening activity similar to α-expansins, however its action is restricted to grass cell walls only. Another feature of the β family of expansins is that it seems to be better represented in monocots as compared to dicots. While only 4 genes have been recognized as β-expansins in Arabidopsis, at least 10 have been identified in rice. Rice also has 11 α-expansins. Sequence analysis to date suggests a two-domain structure. Most notably, the two key aspartate residues thought to function in the enzyme's catalytic site are readily identified as conserved features of both types of expansins (Davies et al. (1995) Biochemistry 34:16210-16220).

The concept that expansins are key endogenous regulators of cell enlargement is now well supported. In addition to being present in actively enlarging tissues, they also seem to mediate adaptive responses to water stress, as well as other stimuli such as associated with gravitropism and phototropism. However, the existence of a large number of expansins in plants suggests that these proteins may play multiple developmental or tissue-specific roles in addition to cell expansion. In fact, the expression profile of a number of expansins is suggestive of their role in processes as diverse as cell wall disassembly during fruit ripening and seed germination, cell separation during abscission, vascular tissue differentiation, and pollen tube penetration (Cosgrove, D. 2000. Nature, 407: 321-326).

Normally expansin is a minor component of the cell wall. Although expansin's physical effect on plant cell walls include rapid induction of wall extension and stimulation of stress relaxation, they do not progressively weaken the cell wall except that the cell wall is longer and thinner after it extends.

Because of the role of expansins in unlocking the network of wall polysaccharides, permitting turgor-driven cell enlargement, novel expansins would be useful for many commercial applications including applications in the lumber, paper and textile industry, and in creating novel transgenic plants.

### SUMMARY OF THE INVENTION

Compositions and methods for modulating plant cell enlargement, plant strength, and plant pliability are provided. In particular, maize expansin polypeptides and the nucleotide sequences encoding such expansins are provided. More specifically, the present invention provides the nucleotide sequence of ZmEXPB4 (SEQ ID NO: 1) and biologically active variants thereof (e.g., SEQ ID NO: 3). Also provided are amino acid sequences (SEQ ID NO: 2 and SEQ ID NO: 4) and biologically active variants thereof encoded by the nucleotide sequences of the invention.

The expansin polypeptides of the invention find use in regulating plant cell enlargement, regulating plant growth, mechanical strength, pliancy and flexibility of the plant cell. The invention reports for the first time that expansins can be used to modulate cell wall strength. Thus, the polypeptides of the invention as well as other expansins find use in modulating, particularly increasing, cell wall strength in transformed plants.

Transformed plants, plant cells, and seeds, as well as methods for making such plants, plant cells, and seeds are additionally provided. It is recognized that a variety of promoters will be useful in the invention, the choice of which will depend in part upon the desired level of expression of the disclosed genes. It is recognized that the levels of expression can be controlled to modulate the levels of expression in the plant as well as to control the developmental expression of the expansins.

### BRIEF DESCRIPTION OF THE DRAWINGS

FIG. 1 depicts an alignment of ZmEXPB4 with 10 expansins from rice (O. sativa). The alignment between the cloned maize β-expansin and all ten beta-expansin genes of rice was performed using Clustal W program. Using BestFit DNA and BestFit Protein Sequence Alignment Analyses, percent identities were calculated both at the nucleotide and amino acid levels, respectively. The β-expansin gene of the invention is 50%, 86%, 83%, 89%, 71%, 83%, 82%, 83%, 82%, and 80% identical, respectively, at nucleotide level with the rice OsExpB1, OsExpB2, OsExpB3, OsExpB4, OsExpB5, OsExpB6, OsExpB7, OsExpB8, OsExpB9, and OsExpB10 genes. At the amino acid level, the percent identity between ZmEXPB4 clone and the rice β-expansin proteins ranged from 49% to 87.5%, the highest being with OsEXPB4.

FIG. 2 describes the gene structure for maize beta-expansin 4. The gene is aligned with an additional ten TUSC generated alleles (Trait Utility System for Corn; see U.S. Pat. No. 5,962,764), showing insertions at different gene regions. Nine out of ten TUSC alleles were found in the beta-expansin 4 gene and one insertion (B08, present in the 3′ UTR) showed sequence variation from the ZmBEXP4 gene.

FIG. 3 is a representation of the differences in expression of ZmBEXP4 in different corn tissues as compiled from the Lynx massively parallel signature sequencing expression profile. This technology is based on matching GATC-17mers tags, in which each cDNA is attached to the surface of a unique microbead. A highly expressed mRNA is represented on a proportionately large number of microbeads. The level of expression of a gene is determined by the abundance of its signature in the total pool of different libraries.

### DETAILED DESCRIPTION OF THE INVENTION

Overview

The present invention provides novel expansin polypeptides and the nucleotide sequences encoding such polypeptides. Expansins are key endogenous regulators of plant cell enlargement. Thus, the compositions of the invention can be used to modulate stress relaxation and extension of cell walls, to stimulate cell enlargement, to modulate growth. Transformed plants having modified cell walls or altered cellular structure may be obtained. By “altered structure” is intended any change in structure or morphology of the transgenic plant when compared to the progenitor untransformed plant. The altered structure is associated with cellular growth and elongation, i.e., plant rigidity, strength, and/or flexibility. Expansins are also implicated in the drought responses of plants, where maintenance of root growth involves increased expansin activity in the growing region. Thus, the compositions can also be used to modulate drought tolerance in plants.

The present invention provides, inter alia, compositions and methods for modulating the total level of proteins of the present invention and/or altering their ratios in a plant. By “modulation” is intended an increase or decrease in a particular character, quality, substance, or response.

By “expansin activity” and “expansin-like activity” is intended the loosening of cell walls, cell enlargement, cell wall disassembly and other cell separation events, as well as cell wall strength. Wall loosening is likely mediated by local disruption of polysaccharide adhesion. Expansins enable the local sliding of wall polymers by reducing adhesion between adjacent wall polysaccharides. Thus, plant mechanical strength and flexibility of the cell wall network of the plant is influenced.

The compositions comprise maize nucleotide and amino acid sequences. Particularly, the nucleotide and amino acid sequence for a maize expansin are provided. This sequence shares homology to conserved expansin genes from rice. As indicated above, expansins are involved in plant cell growth. Generally, expansins are involved in enzymatic wall loosening. While the invention is not bound by any particular mechanism of action, expansins may act to weaken the non-covalent binding between wall polysaccharides, thereby allowing turgor-driven polymer creep. See, McQueen-Mason and Cosgrove (1994) Proc Natl Acad Sci USA 91:6574-6578. The affects of expansin on plant cell walls include rapid induction of wall extension and stimulation of stress relaxation. Expansin movement may be confined to lateral diffusion along the surface of the cellulose microfibril. Such diffusion enables expansin to search the microfibril surface, locally loosening its attachment to the matrix, and allowing chain movement and stress relaxation. See, Cosgrove (2000) Nature 407:321-326, and the references cited therein.

Therefore, the compositions of the invention can be used to modulate stress relaxation and extension of cell walls, to stimulate cell enlargement, to modulate growth. Particularly, the compositions of the invention can be used to modulate mechanical strength, pliability, and flexibility in plants. In one embodiment, the compositions are useful in modulating resistance to brittle snap in plants, particularly maize plants. For example, resistance to brittle snap can be increased by expression or increased expression of the expansin nucleotide sequences of the invention in a plant.

“Brittle snap” or “brittle stalks” are terms used to describe the breakage of corn stalks, for example by severe winds. Corn stalks are more prone to snapping during the rapid elongation stage of growth, especially during the period just prior to tasseling. Rapidly growing corn is subject to breakage from wind as well as other physical phenomenon such as cultivation, hilling, or anhydrous application where stalks are bent by a low tool bar. Thus, expression or overexpression of the expansin sequences of the invention during this period of rapid growth can increase stalk flexibility and increase resistance to brittle snap. By “brittle snap resistance” or “brittle stalk resistance” is intended the ability of the plant to remain intact. In general “brittle stalk resistance” or “brittle snap resistance” is increased by at least 1%, 5%, 10%, 20%, 30%, 40%, 50%, 60%, 70%, 80%, 90% or more relative to a native control plant, plant part or plant cell.

Additionally, the sequences may be used in paper processing as expansin has been shown to weaken cellulose paper. Additionally, the expansins may find use in the paper recycling industry and in the production of virgin paper as the pulp for virgin paper is made by disrupting the bonding between plant fibers. Thus, the expansins of the invention may be used for bioengineering of cell walls, either to manipulate the growth and texture of plants, and/or to modify the structure and physical properties of cell walls used in commercial products such as wood, textiles, and polymers.

In another embodiment, the sequences of the invention may be used to improve forage quality. Down regulation of the sequences of the invention results in brittle plants that may be easier for livestock to eat and digest. In this manner, any means for downregulation of expression can be used including antisense expression and co-suppression. In this embodiment a promoter developmentally expressed only in mature plants or a stalk-preferred promoter would be preferred.

Sequences of the invention, as discussed in more detail below, encompass coding sequences, antisense sequences, and fragments and variants thereof. Expression of the sequences of the invention can be used to modulate or regulate the expression of corresponding expansin proteins. The method involves stably transforming a plant with an expansin nucleotide sequence capable of modulating plant strength and flexibility operably linked with a promoter capable of driving expression of a gene in a plant cell.

It is believed that the invention, for the first time, recognizes that expansins are also involved in modulating cell wall strength. Thus, the invention encompasses the use of any expansin to modulate cell wall strength in plants. A number of expansins are known in the art and can be used in the methods of the invention. See, for example, Cosgrove (2000) Nature 407:321-326 and the references cited therein; Cosgrove (1999) Annu Rev Plant Physiol Plant Mol Biol 50:391-417 and the references cited therein; Shcherban et al. (1995) Proc Natl Acad Sci USA 92:9245-9249; and Cosgrove et al. (1997) Proc Natl Acad Sci USA 94:6559-6564; all of which are herein incorporated by reference. In this manner, known expansins can be used to transform any plant of interest. As discussed in more detail below, a number of promoters may be used to drive expression of the expansin coding sequences.

Compositions

Compositions of the invention include the sequence for a maize expansin nucleotide sequence. In particular, the present invention provides for isolated nucleic acid molecules comprising nucleotide sequences encoding the amino acid sequence shown in SEQ ID NO: 2 or SEQ ID NO: 4. Further provided are polypeptides having an amino acid sequence encoded by a nucleic acid molecule described herein, for example those set forth in SEQ ID NO: 1 or SEQ ID NO: 3, and fragments and variants thereof. Such sequences are referred to herein as “expansin sequences of the invention.”

The invention encompasses isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably culture medium represents less than about 30%, 20%, 10%, or 5% (by dry weight) of chemical precursors or non-protein-of-interest chemicals.

Fragments and variants of the disclosed nucleotide sequences and proteins encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence have expansin-like activity and thereby affect development, developmental pathways, and cellular strength and plant flexibility. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins of the invention.

A fragment of an expansin nucleotide sequence that encodes a biologically active portion of an expansin protein of the invention will encode at least 15, 25, 30, 40, 50, 100, 150, 200, 250, 282 contiguous amino acids, or up to the total number of amino acids present in a full-length expansin protein of the invention (for example, 282 amino acids from SEQ ID NO: 2 or SEQ ID NO: 4). Fragments of an expansin nucleotide sequence that are useful as hybridization probes for PCR primers generally need not encode a biologically active portion of an expansin protein.

Thus, a fragment of an expansin nucleotide sequence may encode a biologically active portion of an expansin protein, or it may be a fragment that can be used as a hybridization probe or PCR primer using methods disclosed below. A biologically active portion of an expansin protein can be prepared by isolating a portion of one of the expansin nucleotide sequences of the invention, expressing the encoded portion of the expansin protein (e.g., by recombinant expression in vitro), and assessing the activity of the encoded portion of the expansin protein. Nucleic acid molecules that are fragments of an expansin nucleotide sequence comprise at least 16, 20, 50, 60, 70, 75, 100, 150, 200, 250, 300, 350, 400, 450, 500, 550, 600, 650, 700, 800, 900, 1000, 1100, 1200, 1300, 1400, 1428 nucleotides, or up to the number of nucleotides present in a full-length expansin nucleotide sequence disclosed herein (for example, 1428 nucleotides for SEQ ID NO: 1 or 2536 nucleotides for SEQ ID NO: 3).

By “variants” is intended substantially similar sequences. For nucleotide sequences, conservative variants include those sequences that, because of the degeneracy of the genetic code, encode the amino acid sequence of one of the expansin polypeptides of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode an expansin protein of the invention. Generally, variants of a particular nucleotide sequence of the invention will have at least about 90%, preferably at least about 91%, 92%, 93%, 94%, 95%, 96%, 97%, and more preferably at least about 98%, 99% or more sequence identity to that particular nucleotide sequence as determined by sequence alignment programs described elsewhere herein using default parameters.

By “variant” protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is they continue to possess the desired biological activity of the native protein, that is, expansin-like activity as described herein. Such variants may result from, for example, genetic polymorphism or from human manipulation. Biologically active variants of a native expansin protein of the invention will have at least about 90%, preferably at least about 91%, 92%, 93%, 94%, 95%, 96%, 97%, and more preferably at least about 98%, 99% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue. Generally expansin variants of the invention will retain the highly conserved cysteines and the His-Phe-Asp motif, as well as the key aspartate residues in the enzyme's catalytic site. See, Davies et al. (1995) Biochemistry 34:16210-16220.

Biological activity of the expansin polypeptides (i.e., influencing plant growth, strength, flexibility and various developmental pathways, including, for example, influencing cell division) can be assayed by any method known in the art. Furthermore, assays to detect expansin-like activity include, for example, cell loosening function and cell wall extension in vitro (Cosgrove et al. (1997) Proc Natl Acad Sci USA 92:6559-6564; Ceccardi et al. (1998) Plant Mol Biol 38:775-783).

The proteins of the invention may be altered in various ways including amino acid substitutions, deletions, truncations, and insertions. Novel proteins having properties of interest may be created by combining elements and fragments of proteins of the present invention as well as other proteins. Methods for such manipulations are generally known in the art. For example, amino acid sequence variants of the expansin proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferred.

Thus, the genes and nucleotide sequences of the invention include both the naturally occurring sequences as well as mutant forms. Likewise, the proteins of the invention encompass both naturally occurring proteins as well as variations and modified forms thereof. Such variants will continue to possess the desired activities. Obviously, the mutations that will be made in the DNA encoding the variant must not place the sequence out of reading frame and preferably will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays.

Variant nucleotide sequences and proteins also encompass sequences and proteins derived from a mutagenic and recombinogenic procedure such as DNA shuffling. With such a procedure, one or more different expansin coding sequences can be manipulated to create a new expansin protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the expansin gene of the invention and other known expansin genes, particularly β-expansin genes to obtain a new gene coding for a protein with an improved property of interest, such as an increased Kₘ in the case of an enzyme. Such shuffling of domains may also be used to assemble novel proteins having novel properties. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:45044509; Crameri et al. (1998) Nature 391:288-291; and U.S. Pat. Nos. 5,605,793 and 5,837,458.

The nucleotide sequences of the invention can be used to isolate corresponding sequences from other organisms, particularly other plants, more particularly other monocots. In this manner, methods such as PCR, hybridization, and the like can be used to identify such sequences based on their sequence homology to the sequences set forth herein. Sequences isolated based on their sequence identity to the entire expansin sequences set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the expansin sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire expansin sequence disclosed herein, or one or more portions thereof, may be used as a probe capable of specifically hybridizing to corresponding expansin sequences and messenger RNAs. To achieve specific hybridization under a variety of conditions, such probes include sequences that are unique among expansin sequences and are preferably at least about 10 nucleotides in length, and most preferably at least about 20 nucleotides in length. Such probes may be used to amplify corresponding sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5M Na ion, typically about 0.01 to 1.0M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than about 24 hours, usually about 4 to 12 hours. Stringent conditions may also be achieved with the addition of destabilizing agents such as formamide. Exemplary low stringency conditions include hybridization with a buffer solution of 30 to 35% formamide, 1M NaCl, 1% SDS (sodium dodecyl sulphate) at 37° C., and a wash in 1× to 2× SSC (20× SSC=3.0M NaCl/0.3M trisodium citrate) at 50 to 55° C. Exemplary moderate stringency conditions include hybridization in 40 to 45% formamide, 1.0M NaCl, 1% SDS at 37° C., and a wash in 0.5× to 1× SSC at 55 to 60° C. Exemplary high stringency conditions include hybridization in 50% formamide, 1M NaCl, 1% SDS at 37° C., and a wash in 0.1× SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10 C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Thus, isolated sequences that encode for an expansin polypeptide and which hybridize under stringent conditions to the expansin sequences disclosed herein, or to fragments thereof, are encompassed by the present invention. Such sequences will be at least about 40% to 50% homologous, about 60%, 65%, or 70% homologous, and even at least about 75%, 80%, 85%, 90%, 91%, 92%, 93%, 94%, 95%, 96%, 97%, 98%, 99% or more homologous with the disclosed sequences. That is, the sequence identity of sequences may range, sharing at least about 40% to 50%, about 60%, 65%, or 70%, and even at least about 75%, 80%, 85%, 90%, 91%, 92%, 93%, 94%, 95%, 96%, 97%, 98%, 99% or more sequence identity.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151 -153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.hlm.nih.gov. Alignment may also be performed manually by inspection.

Unless otherwise stated, sequence identity/similarity values provided herein refer to the value obtained using GAP Version 10 using the following parameters: % identity using GAP Weight of 50 and Length Weight of 3;% similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

GAP uses the algorithm of Needleman and Wunsch (1970) J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1 ,2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman et al. (1970) J. Mol. Biol. 48:443. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The methods of the invention can be used with other methods available in the art for enhancing stalk strength and flexibility in plants. For example, any one of a variety of second nucleotide sequences may be utilized, embodiments of the invention encompass those second nucleotide sequences that, when expressed in a plant, help to modulate stalk strength and flexibility. It is recognized that such second nucleotide sequences may be used in either the sense or antisense orientation depending on the desired outcome. Other plant expansin proteins include those described in U.S. Pat. No. 6,184,440; U.S. Pat. No. 5,959,082, Brummell et al. (1999) Plant Mol Biol 39:161-169; Cho and Kende (1997) Plant Physiol 113:1137-1143; Cho and Kande (1998) Plant J 15:805-812; and Carpita (1996) Annu Rev Plant Physiol Plant Mol Biol 47:445-476; all of which are herein incorporated by reference.

Expression of Sequences

The nucleic acid sequences of the present invention can be expressed in a host cell such as bacteria, yeast, insect, mammalian, or preferably plant cells. It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous nucleotide sequence can be from a species different from that from which the nucleotide sequence was derived, or, if from the same species, the promoter is not naturally found operably linked to the nucleotide sequence. A heterologous protein may originate from a foreign species, or, if from the same species, is substantially modified from its original form by deliberate human intervention.

By “host cell” is meant a cell, which comprises a heterologous nucleic acid sequence of the invention. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize host cell.

As used herein, the term plant includes plant cells, plant protoplasts, plant cell tissue cultures from which maize plants can be regenerated, plant calli, plant clumps, and plant cells that are intact in plants or parts of plants, such as embryos, pollen, ovules, seeds, flowers, kernels, ears, cobs, leaves, husks, stalks, roots, root tips, anthers, silk and the like.

The expansin sequences of the invention are provided in expression cassettes or DNA constructs for expression in the plant of interest. Likewise, known expansins may also be provided in expression cassettes where the expansin is used to modify cell wall strength in a transformed plant. The cassette will include 5′ and 3′ regulatory sequences operably linked to an expansin sequence of the invention. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the expansin sequence to be under the transcriptional regulation of the regulatory regions. The expression cassette may additionally contain selectable marker genes.

The expression cassette will include in the 5′-3′ direction of transcription, a transcriptional and translational initiation region, an expansin DNA sequence of the invention, and a transcriptional and translational termination region functional in plants. The transcriptional initiation region, the promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a chimeric gene comprises a coding sequence operably linked to a transcription initiation region that is heterologous to the coding sequence.

While it may be preferable to express the sequences using heterologous promoters, the native promoter sequences may be used. Such constructs would change expression levels of expansin in the host cell (i.e., plant or plant cell). Thus, the phenotype of the host cell (i.e., plant or plant cell) is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet 262:141-144; Proudfoot (1991) Cell 64:671-674; Sanfacon et al. (1991) Genes Dev. 5:141-149; Mogen et al. (1990) Plant Cell 2:1261-1272; Munroe. et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed plant. That is, the genes can be synthesized using plant-preferred codons for improved expression. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) PNAS USA 86:6126-6130); potyvirus leaders, for example, TEV leader (Tobacco Etch Virus) (Allison et al. (1986); MDMV leader (Maize Dwarf Mosaic Virus); Virology 154:9-20), and human immunoglobulin heavy-chain binding protein (BiP), (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp.177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother 36:913-919; Hlavka et al. (1985) Handbook of Experimental Pharmacology, Vol. 78 ( Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

A number of promoters can be used in the practice of the invention. The promoters can be selected based on the desired outcome. That is, the nucleic acids can be combined with constitutive, tissue-preferred, or other promoters for expression in the host cell of interest. Such constitutive promoters include, for example, the core promoter of the Rsyn7 (copending U.S. application Ser. No. 08/661,601); the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet 81:581-588); MAS (Velten et al. (1984) EMBO J. 3:2723-2730); ALS promoter (U.S. application Ser. No. 08/409,297), and the like. Other constitutive promoters include, for example, U.S. Pat. Nos. 5,608,149; 5,608,144; 5,604,121; 5,569,597; 5,466,785; 5,399,680; 5,268,463; and 5,608,142.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2): 247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expansin expression within a particular plant tissue. Tissue-preferred promoters include Yamamoto et al. (1997) Plant J. 12(2): 255-265; Kawamata et al. (1997) Plant Cell Physiol. 38(7): 792-803; Hansen et al. (1997) Mol. Gen Genet. 254(3): 337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascini et al. (1996) Plant Physiol. 112(2): 513-524; Yamamoto et al. (1994) Plant Cell Physiol. 35(5): 773-778; Lam (1994) Results Probl. Cell Differ 20:181-196; Orozco et al. (1993) Plant Mol Biol. 23(6): 1129-1138; Matsuoka et al. (1993) Proc Natl. Acad. Sci. USA 90(20): 9586-9590; and Guevara-Garcia et al. (1993) Plant J. 4(3): 495-505.

Leaf-specific promoters are known in the art. See, for example, Yamamoto et al. (1997) Plant J. 12(2): 255-265; Kwon et al. (1994) Plant Physiol. 105:357-67; Yamamoto et al. (1994) Plant Cell Physiol. 35(5): 773-778; Gotor et al. (1993) Plant J. 3:509-18; Orozco et al. (1993) Plant Mol. Biol. 23(6): 1129-1138; and Matsuoka et al. (1993) Proc. Natl. Acad. Sci. USA 90(20): 9586-9590.

Promoters expressed during rapid plant growth and elongation can also be used. Such developmentally regulated promoters include, but are not limited to, the A. thaliana RPL16B promoter (Williams et al. (1995) Plant J. 8:65-76) and the R. communis L. PLD promoter (Xu et al. (1997) Plant Phys. 115:387-395. Such promoters would allow for overexpression of the expansin sequences at developmental stage that has relevance to the brittle snap phenomenon.

Promoters expressed only in later developmental stages may also be used in the invention. Likewise, stalk-preferred promoters, such as F3.7 (Baszczynski et al. (1997) Maydica 42:189-201, herein incorporated by reference) may be used.

One of skill in will recognize that after the recombinant expression cassette is stably incorporated in transgenic plants and confirmed to be operable, it can be introduced into other plants by sexual crossing. Any of a number of standard breeding techniques can be used, depending upon the species to be crossed. In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self crossed to produce a homozygous inbred plant. The inbred plant produces seed containing the newly introduced heterologous nucleic acid. These seeds can be grown to produce plants that would produce the selected phenotype. Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences.

The method of transformation/transfection is not critical to the instant invention; various methods of transformation or transfection are currently available. As newer methods are available to transform crops or other host cells they may be directly applied. Accordingly, a wide variety of methods have been developed to insert a DNA sequence into the genome of a host cell to obtain the transcription and/or translation of the sequence to effect phenotypic changes in the organism. Thus, any method, which provides for effective transformation/transfection may be employed.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat No. 5,563,055 and Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In vitro Cell Dev. Biol. 27P: 175-182 (soybean); Singh et al. (1998) Theor AppL. Genet 96:319-324 (soybean); Datta et al. (1990) Biotechnology 8:736-740 (rice); Klein et al. (1988) Proc. Natl. Acad. Sci. USA 85:43054309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

The present invention may be used for transformation of any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:4057) and the lambda derived P L promoter and N-gene ribosome binding site (Shimatake et al. (1981) Nature 292:128). Examples of selection markers for E. coli include, for example, genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva et al. (1983) Gene 22:229-235 and Mosbach et al. (1983) Nature 302:543-545).

A variety of eukaryotic expression systems such as yeast, insect cell lines, plant and mammalian cells, are known to those of skill in the art. As explained briefly below, a polynucleotide of the present invention can be expressed in these eukaryotic systems. In some embodiments, transformed/transfected plant cells, as discussed infra, are employed as expression systems for production of the proteins of the instant invention.

Synthesis of heterologous nucleotide sequences in yeast is well known. Sherman, F., et al. (1982) Methods in Yeast Genetics, Cold Spring Harbor Laboratory is a well recognized work describing the various methods available to produce the protein in yeast. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lists. The monitoring of the purification process can be accomplished by using Western blot techniques or radioimmunoassay of other standard immunoassay techniques.

The sequences of the present invention can also be ligated to various expression vectors for use in transfecting cell cultures of, for instance, mammalian, insect, or plant origin. Illustrative cell cultures useful for the production of the peptides are mammalian cells. A number of suitable host cell lines capable of expressing intact proteins have been developed in the art, and include the HEK293, BHK21, and CHO cell lines. Expression vectors for these cells can include expression control sequences, such as an origin of replication, a promoter (e.g. the CMV promoter, a HSV tk promoter or pgk (phosphoglycerate kinase) promoter), an enhancer (Queen et al. (1986) Immunol. Rev. 89:49), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection.

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (See, Schneider, J. Embryol. Exp. Morphol. 27:353-365 (1987).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague, et al. (1983) J. Virol. 45:773-781). Additionally, gene sequences to control replication in the host cell may be incorporated into the vector such as those found in bovine papilloma virus type-vectors. Saveria-Campo, M., (1985) Bovine Papilloma Virus DNA a Eukaryotic Cloning Vector in DNA Cloning Vol. II a Practical Approach, D. M. Glover, Ed., IRL Press, Arlington, Va.pp. 213-238.

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextrin, electroporation, biolistics, and micro-injection of the DNA directly into the cells. The transfected cells are cultured by means well known in the art. Kuchler, R. J. (1997) Biochemical Methods in Cell Culture and Virology, Dowden, Hutchinson and Ross, Inc.

It is recognized that with these nucleotide sequences, antisense constructions, complementary to at least a portion of the messenger RNA (mRNA) for the expansin sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity. See, U.S. Pat. Nos. 5,283,184 and 5,034,323; herein incorporated by reference.

In some embodiments, the content and/or composition of polypeptides of the present invention in a plant may be modulated by altering, in vivo or in vitro, the promoter of the nucleotide sequence to up- or down-regulate expression. For instance, an isolated nucleic acid comprising a promoter sequence is transfected into a plant cell. Subsequently, a plant cell comprising the promoter operably linked to a polynucleotide of the present invention is selected for by means known to those of skill in the art such as, but not limited to, Southern blot, DNA sequencing, or PCR analysis using primers specific to the promoter and to the gene and detecting amplicons produced therefrom. A plant or plant part altered or modified by the foregoing embodiments is grown under plant forming conditions for a time sufficient to modulate the concentration and/or composition of polypeptides of the present invention in the plant. Plant forming conditions are well known in the art and discussed briefly, supra.

In general, concentration or composition is increased or decreased by at least 5%, 10%, 20%, 30%, 40%, 50%, 60%, 70%, 80%, or 90% relative to a native control plant, plant part, or cell lacking the aforementioned recombinant expression cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra. Induction of expression of a polynucleotide of the present invention can also be controlled by exogenous administration of an effective amount of inducing compound. Inducible promoters and inducing compounds, which activate expression from these promoters, are well known in the art. In preferred embodiments, the polypeptides of the present invention are modulated in monocots, particularly maize.

The following examples are offered by way of illustration and not by way of limitation.

### EXPERIMENTAL

### Example 1. Cloning of a brittle stalk gene

Cloning of maize genes that are known exclusively by their mutant phenotypes continues to be a challenging proposition. The only technique that has worked to any degree is transposon tagging. While it has allowed maize researchers to clone several genes over the last decade, the risk factor associated with transposon tagging is abnormally high, causing a high proportion of projects to end in vain. One reason for this is that typically a single gene is selected as a tagging and cloning target. But if the number of targets is increased, the effectiveness and success rate of a tagging project can be increased significantly. For example, it was demonstrated that when pollen from Mangelsdorf tester, which is homozygous recessive at ten different loci, was used to pollinate plants active in Mutator (Mu, a transposable element system in maize), mutants defective in each of these 10 genes appeared in the same F1 progeny (Johal, G. S. and Gruis, D. 1995. MNL, 69: 47-48).

While this approach using multi-gene testers works quite effectively, the difficulty of developing such stocks seriously hinders the usefulness of the approach. An attractive alternative, however, is provided by B-A translocations, stocks of which are available at the maize Co-Op for 19 of the 20 chromosome arms of the maize complement. One key feature of B-A translocations is that a chromosome carrying the B centromere very frequently undergoes nondisjunction at the second pollen division during male gametophyte development (Roman, H. 1947. Genetics, 32: 391-409). This results in the formation of one sperm cell with two B-A chromosomes (hyperploid) and the other with none (hypoploid). Since the function and growth of the male gametophyte is controlled by the balanced vegetative nucleus (Roman, H. 1948. PNAS, 34: 36-42), both the hyperploid and hypoploid gametes function normally during fertilization. However, when the hypoploid sperm nucleus fertilizes an egg cell, the resulting offspring (kernel) lacks all paternal genes that are localized on the deficient chromosome arm.

This ability of B-A translocations to produce hemizygous offspring has been utilized very effectively for determining, in a single generation (F1), the chromosome arm on which recessive mutations are located. This feature of B-A translocations can also be utilized for tagging and cloning multiple genes at a time. For example, when Mu-active plants are fertilized with deficient pollen from B-A stocks, any Mu-insertion in a maternal gene that happens to reside on the deficient chromosome arm may result in a mutant phenotype in the F1. Thus mutations in many genes, present on the same arm of a chromosome, can be uncovered in a single generation and can be identified by appropriate screening protocols. Moreover, this approach also provides a straightforward procedure for cloning the tagged gene(s). For example, once a mutant is found, its DNA can be used directly to clone the responsible gene. A Southern blot analysis, in which DNA from the mutant is compared with that of its wild-type siblings for Mu element polymorphism, can be employed to identify the Mu element that may have caused the mutation. Alternatively, a PCR approach can also be used to perform such co-segregation analysis. Many protocols are available that allow specific amplification of the maize chromosomal DNA immediately flanking Mu insertional sites and any can be used in the method.

The method has been used to clone a maize expansin gene. The gene may be used to manipulate brittle snap resistance of maize, a problem plaguing many of the maize hybrids grown especially in Northern Plains areas. While the example demonstrates the cloning of a particular gene, this can be used to identify and clone any gene of interest.

Materials:

The brittle stalk mutant has been designated bk*-dm. It appeared in a cross of a Mu-active line with TB-9Lc (a B-A translocation involving about 90% of the long arm of chromosome 9). Progeny from 73 F1 ears, involving different Mu-active stocks and TB-9Lc were planted and manually screened for plants having brittle leaves. The bk*-dm mutant was identified as a single segregant out of a population of 436 plants derived from a single F1 ear, suggesting that it may have originated as a result of a new Mu transposition in a locus on 9L. Two previously known brittle plant mutations, bk1 and bk3, have already been shown to be localized to 9L (Brewbaker, 1995, MNL, 69: 58-59; Langham, 1940. MNL, 14: 21-22). Like these mutants, leaves of bk*-dm were highly brittle and readily snapped when pressed between fingers. All other parts of the plant, such as the stalk and brace roots, were also brittle and easily breakable.

Cloning and gene identity:

To clone the gene defective in bk*-dm, a Southern-blot based co-segregation analysis was performed. DNA was extracted from bk*-dm as well as from 32 wild-type (WT) siblings of this mutant. Next, DNA from bk*-dm and pooled DNA of its WT siblings was digested with 7 different restriction enzymes, electrophoresed side by side and blotted onto nylon membranes. The resulting blots were hybridized with probes from 9 different Mu elements. This polymorphism analysis yielded a Mu8-hybridizing, 3.8 kb/SaII fragment that was present in the bk*-dm mutant but absent in the WT siblings pool. Southern blot analysis employing DNA from individual WT siblings confirmed the exclusive presence of the 3.8 kb restriction fragment in bk*-dm. To clone this SaII fragment, a size-fractionated library was prepared in Zap II, a Lambda based cloning vector from Stratagene. The cloned fragment was subcloned by in vivo excision and sequenced using T3 and T7 primers of the cloning vector and a terminal inverted repeat (TIR)-specific primer from Mu. BLAST analysis of the sequence showed that our clone harbors a complete gene whose sequence matches the best with β-expansin4 (ExpB4) of rice, which is reported to contain at least 10 different β-expansin genes. The GenBank Acc. Nos. for the rice β-expansin genes include:

OsExpB1 (GenBank Acc. No. AF261270), OsExpB2 (GenBank Acc. No. U95968), OsExpB3 (GenBank Acc. No. AF261271), OsExpB4 (GenBank Acc. No. AF261272), OsExpB5 (GenBank Acc. No. AF261273), OsExpB6 (GenBank Acc. No. AF261274), OsExpB7 (GenBank Acc. No. AF261275), OsExpB8 (GenBank Acc. No. AF261276), OsExpB9 (GenBankAcc. No. AF261277), and OsExpB10 (GenBank Acc. No. AF261278).

The Mu8 element responsible for the brittle phenotype of bk*-dm is inserted about 260 bp upstream of the initiation codon (ATG) of ExpB4. A full length EST (cpibl68r, ˜1.5 kb) corresponding to ExpB4 was found in a Pioneer proprietary library (made from immature ear). It showed 89.2% identity at nucleotide level and 87.5% identity at amino acid level with ExpB4 of rice.

In order to confirm that the brittle phenotype of bk*-dm is in fact due to a mutation in ExpB4, a reverse genetic approach was employed to ask if any of the brittle plant mutants that were in the collection also had a Mu insertion in the ExpB4 gene. Many brittle plant mutations have been generated over the years using both the directed and random mutagenesis approaches involving Mu. So, while some of these brittle stalk mutants were known to be due to defects in genes like bk1 and bk2, there were others whose identity in terms of locus connection had not been investigated. One such mutant was bk*-gj. A Mu-specific PCR approach was used to detect the probable presence of a Mu insertion in the ExpB4 gene of these mutants. Two ExpB4 specific primers were designed and used one by one in combination with a Mu-TIR specific primer in a PCR protocol in which the template DNA came from a number of brittle plant mutants.

The ExpB4 specific forward primer:

SEQ ID NO: 5 5′-ACCACCCACCCACCACTGCTCGCACTC-3′

The ExpB4 specific reverse primer from the exon-1:

SEQ ID NO:6 5′CCTTCTGCAGGGGCCCCAGTACACGG-3′

Used in combination with the Mu-TIR specific primer,

SEQ ID NO:9 5′-CGCCMCGCCTCCATTTCGTCGAATCC-3′

An ExpB4-hybridizable PCR product was amplified from the bk*-gj DNA. This PCR-amplified product was cloned in TOPO (a cloning vector from Invitrogen) and sequenced using M13 forward and reverse primers. Sequence analysis revealed that a Mu element had inserted in bk*-gj about 90 bp upstream of the initiator codon of the ExpB4 gene. This location is different than the Mu8 insertional site in bk*-dm, clearly indicating that these two brittle plant mutations had independent origins. In maize, when two (or more) independent mutational events within a few hundred base pairs of each other result in an identical mutant phenotype, this is considered a proof for the identity of the mutant gene. Thus, the function of ExpB4 is to specifically provide mechanical strength and pliancy to the maize plant. This revelation provides direct evidence, for the first time, for the biological role of an expansin in a plant.

Usefulness of the B-A translocation technique in cloning maize genes

There are two key advantages of this cloning approach. The first is the tagging of multiple genes in a single experiment—and that too in a chromosome arm-specific way. With the exception of 8S, B-A translocations exist for all maize chromosome arms, and together, they are expected to uncover as much as 80% of the genome. Given the fact that break points for many of these lie close to centromeres, the proportion of genes uncovered by these translocations may be even higher than 80%. So, generally any gene that results in an observable or measurable phenotype when mutated can be cloned using the B-A approach.

The second advantage is the ease and rapid rate of cloning, which can be attempted as soon as the mutant is identified. There is no need to propagate the mutant further for the purpose of co-segregation analysis, the way it is normally done with traditional transposon tagging techniques. The method is also amenable to applying faster PCR-based approaches for identifying and cloning the relevant gene by co-segregation analysis.

There are a few advantages of this technique. For example, genes whose function is absolutely required for the development of the male gametophyte can only be tagged and cloned by this method. Mutations in such genes are normally male non-transmissible, and thus refractory to genetic analysis. This is not a problem with B-A translocations, because the deficiency only develops during sperm cell genesis and not during the division that gives rise to the vegetative nucleus, the functional curator of the male gametophyte. Additionally, since the tagged genes can be cloned from the F1 material, it does not matter whether the mutant is lethal or sterile. All that is required is some DNA from the mutant. Furthermore, if there is a need to propagate the mutant further, no genetic or molecular marker is required to follow the inheritance of the tagged mutant allele, as this would be the only allele that would transmit through pollen.

### Example 2. Sequence Analysis of the Maize Expansin Sequence Gene Structure of ZmExpB4:

The β-expansin 4 gene of maize (ZmEXPB4; SEQ ID NO: 3) has four exons and three introns. The size of exons 1 through 4 is 211 bp (265 bp to 475 bp); 110 bp (592 bp to 701 bp); 185 bp (1128 bp to 1312 bp); and 343 bp (1698 bp to 2040 bp), respectively. Intron 1 is 116 bp long, intron 2, 426 bp, and intron 3, 385 bp long. The length of the complete ZmEXPB4 cDNA is 1389 bp (SEQ ID NO: 1), while only 889 of these nucleotides are utilized to encode the ZmEXPB4 protein (SEQ ID NO: 2). ZmEXPB4 cDNA is comparable in size to beta-expansins of rice, which range from 933 bp to 1319 bp in length.

Multiple Alignment of beta-expansins:

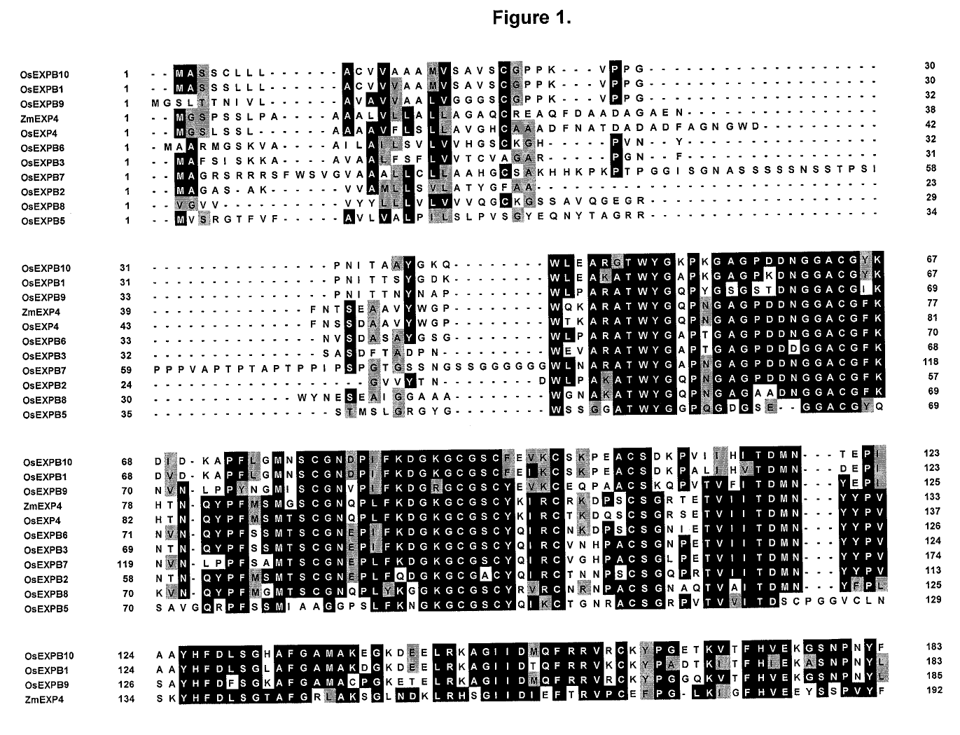

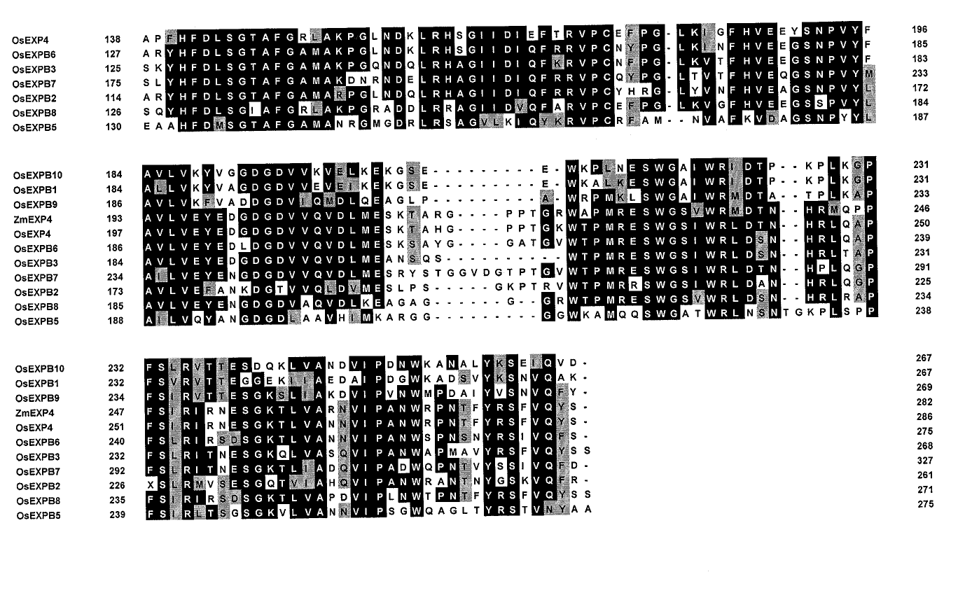

So far ten β-expansins have been characterized in rice (Shcherban, T. Y. et al. 1995. PNAS, 92: 9245-9249; and Cosgrove, D. J. 1997. PNAS, 94: 6559-6564), and most of these genes are quite homologous at the 3′ end (FIG. 1). Multiple alignment between the cloned maize β-expansin and all ten beta-expansin genes of rice was performed using Clustal W program. Using BestFit DNA and BestFit Protein Sequence Alignment Analyses, percent identities were calculated both at the nucleotide and amino acid levels, respectively. Our β-expansin gene is 50%, 86%, 83%, 89%, 71%, 83%, 82%, 83%, 82%, and 80% identical, respectively, at nucleotide level with the rice OsExpB1 (GenBank Acc. No. AF261270), OsExpB2 (GenBank Acc. No. U95968), OsExpB3 (GenBank Acc. No. AF261271), OsExpB4 (GenBank Acc. No. AF261272), OsExpB5 (GenBank Acc. No. AF261273), OsExpB6 (GenBank Acc. No. AF261274), OsExpB7 (GenBank Acc. No. AF261275), OsExpB8 (GenBank Acc. No. AF261276), OsExpB9 (GenBank Acc. No. AF261277), and OsExpB10 (GenBank Acc. No. AF261278) genes. At the amino acid level, the percent identity between our clone and the rice β-expansin proteins ranged from 49% to 87.5%, the highest being with OsEXPB4 (FIG. 1). Based on this sequence identity, we have designated the maize β-expansin gene as ZmExpB4.

The invention provides the ability to modulate plant cell wall strength and pliability by using any expansin, including the novel expansins disclosed herein. For the first time, the biological function of an expansin is identified as mediating cell wall strengthening. Having recognized this ability, any expansin can be used to modulate cell wall strength in any plant of interest.

### Example 3. Transformation and Regeneration of Transgenic Plants in Maize

Immature maize embryos from greenhouse donor plants are bombarded with a plasmid containing an expansin nucleotide sequence operably linked to a ubiquitin promoter plus a plasmid containing the selectable marker gene PAT (Wohlleben et al. (1988) Gene 70:25-37) that confers resistance to the herbicide Bialaphos. Transformation is performed as follows. All media recipes are in the Appendix.

Preparation of Target Tissue

The ears are surface sterilized in 30% Chlorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

A plasmid vector comprising the expansin nucleotide sequence operably linked to a ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in TrisEDTA buffer (1 μg total)

100 μl 2.5M CaCl₂

10 μl 0.1M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing pofting soil and grown for 1 week in a growth chamber, subsequently grown an additional 1-2 weeks in the greenhouse, then transferred to classic 600 pots (1.6 gallon) and grown to maturity. Plants are monitored and scored for altered expansin activity.

Bombardment and Culture Media

Bombardment medium (560Y) comprises 4.0 g/l N6 basal salts (SIGMA C-1416), 1.0 ml/l Eriksson's Vitamin Mix (1000X SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000X SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 4. Agrobacterium-mediated Transformation in Maize

For Agrobacterium-mediated transformation of maize with an expansin nucleotide sequence of the invention operably linked to a ubiquitin promoter, preferably the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the DNA construct containing the expansin nucleotide sequence to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants.

### Example 5. Sunflower Meristem Tissue Transformation

Sunflower meristem tissues are transformed with an expression cassette containing the expansin sequence operably linked to a ubiquitin promoter as follows (see also European Patent Number EP 0 486233, herein incorporated by reference, and Malone-Schoneberg et al. (1994) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer et al. (Schrammeijer et al. (1990) Plant Cell Rep. 9: 55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige et al. (1962) Physiol. Plant, 15: 473-497), Shepard's vitamin additions (Shepard (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA3), pH 5.6, and 8 g/l Phytagar.

The explants are subjected to microprojectile bombardment prior to Agrobacterium treatment (Bidney et al. (1992) Plant Mol. Biol. 18: 301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 is used in all transformation experiments. A binary plasmid vector comprising the expression cassette that contains the expansin gene operably linked to the ubiquitin promoter is introduced into Agrobacterium strain EHA105 via freeze-thawing as described by Holsters et al. (1978) Mol. Gen. Genet 163:181-187. This plasmid further comprises a kanamycin selectable marker gene (i.e, nptll). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH4CI, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for expansin-like activity.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by expansin activity analysis of leaf extracts while transgenic seeds harvested from NPTII-positive To plants are identified by expansin activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox bleach solution with the addition of two to three drops of Tween 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bactopeptone, and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD 600. Particle-bombarded explants are transferred to GBA medium (374E), and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for expansin activity using assays known in the art. After positive (i.e., for expansin expression) explants are identified, those shoots that fail to exhibit expansin activity are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for expansin expression are grafted to Pioneer hybrid 6440 in vitro-grown sunflower seedling rootstock. The rootstocks are prepared in the following manner. Seeds are dehulled and surface-sterilized for 20 minutes in a 20% Clorox bleach solution with the addition of two to three drops of Tween 20 per 100 ml of solution, and are rinsed three times with distilled water. The sterilized seeds are germinated on the filter moistened with water for three days, then they are transferred into 48 medium (half-strength MS salt, 0.5% sucrose, 0.3% gelrite pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

### Example 6

This example describes chromosomal allele site determination for ZmEXPB4 by PCR and Southern Blot.

The polymerase chain reaction protocol consists of preparing denatured DNA. Then the single stranded preparation is annealed to short primer sequences complementary to sites on each side of the target region. DNA polymerase is used to synthesize a single strand form the 3′ end of each primer. The entire cycle is repeated and the given sequence is amplified. These amplified nucleic acid strands can then be loaded on an agarose gel where they are separated by size during an electrophoresis procedure. Following electrophoresis, the DNA is transferred by southern blot to nitrocellulose. The immobilized DNA is then hybridized with a complementary radioactive probe, which can be measured by autoradiography. Each complementary sequence gives rise to a labeled band at a position determined by the size of the DNA fragment.

In order to confirm the chromosomal location of ZmEXPB4, two gene specific primers were designed which would amplify a fragment of approximately 282 base pairs only from the 5′ end of the gene.

For amplifying the 282 base pair fragment the following primers were used:

A forward primer:

SEQ ID NO: 5 5′-ACCACCCACCCACCACTGCTCGCACTC-3′

designed from 125 base pair upstream of the ATG site of the ZmBEXP4 gene, used in combination with a reverse primer from the exon -1:

SEQ ID NO:6 5′-CCTTCTGCAGGGGCCCCAGTACACGG-3′

These primers were used to amplify gene specific fragments using template DNA from maize-oat addition lines along with maize and oat parents. The PCR amplification results showed that ZmEXPB4 had two copies in maize, one each located on chromosome 1 and 9. Furthermore, the intensity of amplified bands and their hybridization signals under very high stringent hybridization and washing conditions indicated that these copies were not divergent from each other. One allele is present on chromosome 9 (ZmEXPB4-1) and the other on chromosome 1 (ZmEXPB4-2).

### Example 7

This example describes the procedure utilized to link the ZmEXPB4 gene with its phenotype by utilizing Mutator (Mu)-inserted mutant alleles.

The Trait Utility System for Corn (TUSC; see U.S. Pat. No. 5,962,764) is a method that employs genetic and molecular techniques to facilitate the study of gene function in maize. Studying gene function implies that the gene's sequence is already known, thus the method works in reverse: from sequence to phenotype. This kind of application is referred to as “reverse genetics”, which contrasts with “forward” methods (such as transposon tagging) that are designed to identify and isolate the gene(s) responsible for a particular trait (phenotype).

Pioneer Hi-Bred International, Inc., has a proprietary collection of maize genomic DNA from approximately 42,000 individual F₁ plants (Reverse genetics for maize., Meeley, R and Briggs, S, 1995, MNL, 69: 67,82). The genome of each of these individuals contains multiple copies of the transposable element family, Mutator (Mu). The Mu family is highly mutagenic; in the presence of the active element Mu-DR, these elements transpose throughout the genome, inserting into genic regions, and often disrupting gene function. By collecting genomic DNA from a large number of individuals, Pioneer has assembled a library of the mutagenized maize genome. Mu insertion events are predominately heterozygous so, given the recessive nature of most insertional mutations, the F₁ plants appear wild-type. Each of the plants was selfed to produce F₂ seed, which was collected. In generating the F₂ progeny, insertional mutations segregate in a Mendelian fashion so are useful for investigating a mutant allele's effect on the phenotype. The TUSC system has been successfully used by a number of laboratories to identify the function of a variety of genes (Cloning and characterization of the maize An1 gene, Bensen, R. J. et al., 1995, Plant Cell 7:75-84; Diversification of C-function activity in maize flower development, Mena, M. et al., 1996, Science 274:1537-1540; Analysis of a chemical plant defense mechanism in grasses, Frey, M. et al., 1997, Science 277:696-699; The control of maize spikelet meristem fate by the APETALA2-like gene Indeterminate spikelet 1, Chuck, G., et al., 1998, Genes & Development 12:1145-1154; A SecY homologue is required for the elaboration of the chloroplast thylakoid membrane and for normal chloroplast gene expression, Roy, L. M. et al., 1998, J. Cell Biol. 141:1-11).

In this example, the TUSC approach was used to generate additional Mu inserted mutant alleles. Ten TUSC generated alleles were provided by using gene specific primers from ZmEXPB4.

The primers utilized for isolation of TUSC mutant alleles included:

a forward primer designated from 70 base pair upstream of the TATA box,

SEQ ID NO:7 5′-TGCTAACTGCGTACACACACAGACCTCT-3′

and a reverse primer from the 3′ UTR,

SEQ ID NO:8 5′-TCAACCCTGCACAGGGGTGTGAAC-3′

used in combination with TIR primer,

SEQ ID NO:9 5′-CGCCAACGCCTCCATTTCGTCGAATCC-3′

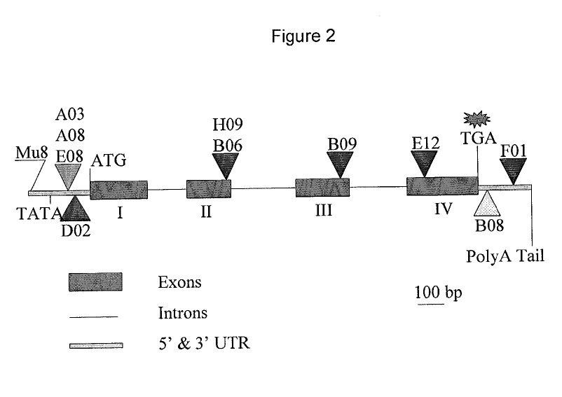

PCR products of these alleles covering the full-length of the gene were amplified, cloned and sequenced. Based on the sequence information, positions of these ten TUSC alleles have been marked on the ZmEXPB4 gene structure (FIG. 2). Nine out of ten TUSC alleles were found in the beta-expansin 4 gene. One insertion, B08, present in the 3′UTR, showed sequence variation from the ZmBEXP4 gene, particularly in the intron-2, intron-3, exon-4 and at the 3′UTR. A specific sequence location having multiple allele insertions was identified in the 5′ UTR of the gene (between the TATA box and ATG) with four insertions in the vicinity of 15 bp were detected. Three exonic insertions, one each in the exon-2, exon-3, and exon-4, were isolated. Insertion B06 was found at the junction of exon-2 and intron-2. Two insertions (B08 and F01) were present in the 3′ UTR (between the stop sign and the poly A tail).

Plants containing insertions are then fingerprinted by using Southern Blot and PCR analyses. Plants characterized as homozygous for the mutant alleles are thereby isolated.

### Example 8

This example describes the method for determining expression of the expansin gene in various tissues.

Expression of ZmEXPB4 gene in different corn tissues was studied by Lynx-massively parallel signature sequencing (MPSS) expression profiles, which is based on matching GATC-17 mers tags (Brenner, et al., 2000. Nat Biotechnology, 18:630-634; and Brenner et al., 2000, PNAS, 97: 1665-1670).

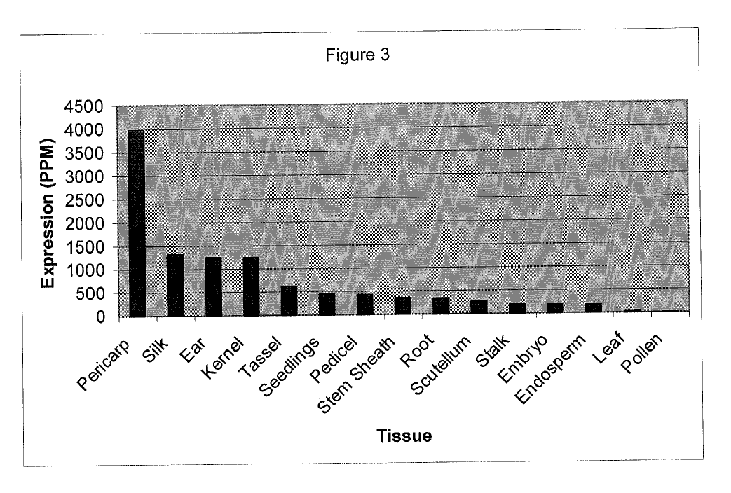

With this technique, each cDNA is attached to the surface of a unique microbead. A highly expressed mRNA is represented on a proportionately large number of microbeads. The level of expression of a gene is determined by the abundance of its signature in the total pool of different libraries. FIG. 3 shows the expression of ZmEXPB4 in different corn tissues as compiled from the Lynx Database. High levels of ZmEXPB4 expression was found in pericarp (3989 ppm), which was 3 times more than the expression found in silk (1320 ppm), ear (1246 ppm), and kernel (1242 ppm) tissues. Moderately high to low expression level was observed in tassel, seedling, pedicel, stem sheath, root, scutellum, pollen and stalk tissues.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. An isolated polypeptide having expansin activity comprising an amino acid sequence selected from the group consisting of:
(a) a polypeptide sequence comprising the amino acid sequence set forth in SEQ ID NO: 2 or SEQ ID NO: 4;
(b) a polypeptide having at least 90% identity to the sequences of (a), wherein said polypeptide retains expansin activity;
(c) a polypeptide encoded by a nucleotide sequence that hybridizes under stringent conditions to a nucleotide sequence comprising the sequence set forth in SEQ ID NO: 1 or SEQ ID NO: 3; and,
(d) a polypeptide sequence comprising at least 40 consecutive amino acids of SEQ ID NO: 2 or SEQ ID NO: 4, wherein said polypeptide retains expansin activity.

2. An isolated nucleotide sequence that encodes a polypeptide having expansin activity, said nucleotide sequence comprising a member selected from the group consisting of:
(a) a nucleotide sequence comprising the sequence set forth in SEQ ID NO: 1 or SEQ ID NO: 3;
(b) a nucleotide sequence encoding a polypeptide comprising the amino acid sequence set forth in SEQ ID NO: 2 or SEQ ID NO: 4;
(c) a nucleotide sequence comprising at least 70 contiguous nucleotides of a sequence of (a) or (b), wherein said sequence encodes a polypeptide having expansin activity;
(d) a nucleotide sequence having at least 90% identity with the nucleotide sequences of (a), (b), or (c), wherein said sequence encodes a polypeptide having expansin activity;
(e) a nucleotide sequence comprising a complementary sequence corresponding to the sequence of (a), (b), (c), or (d); and
(f) a nucleotide sequence that hybridizes under stringent conditions to the nucleotide sequences of (a), (b), (c), (d), or (e) wherein said sequence encodes a polypeptide having expansin activity.

3. A DNA construct comprising a nucleotide sequence of claim 2, wherein said nucleotide sequence is operably linked to a promoter that drives expression in a plant cell.

4. An expression vector comprising the DNA construct of claim 3.

5. A host cell having stably incorporated into its genome at least one nucleotide sequence, wherein said nucleotide sequence is operably linked to a promoter that drives expression in the host cell, wherein said nucleotide sequence is selected from the group consisting of:
(a) a nucleotide sequence comprising the sequence set forth in SEQ ID NO: 1 or SEQ ID NO: 3;
(b) a nucleotide sequence encoding a polypeptide comprising the amino acid sequence set forth in SEQ ID NO: 2 or SEQ ID NO: 4;
(c) a nucleotide sequence comprising at least 70 contiguous nucleotides of a sequence of (a) or (b), wherein said sequence encodes a polypeptide having expansin activity;
(d) a nucleotide sequence having at least 60% identity with the nucleotide sequences of (a), (b), or (c), wherein said sequence encodes a polypeptide having expansin activity;
(e) a nucleotide sequence comprising a complementary sequence corresponding to the sequence of (a), (b), (c), or (d); and
(f) a nucleotide sequence that hybridizes under stringent conditions to the nucleotide sequences of (a), (b), (c), (d), or (e), wherein said sequence encodes a polypeptide having expansin activity.

6. The host cell of claim 5, wherein said cell is a plant cell.

7. A plant or parts thereof, having stably incorporated into its genome at least one nucleotide sequence, said nucleotide sequence operably linked to a promoter that drives expression in a plant cell, wherein said nucleotide sequence is selected from the group consisting of:
(a) a nucleotide sequence comprising the sequence set forth in SEQ ID NO: 1 or SEQ ID NO: 3;
(b) a nucleotide sequence encoding a polypeptide comprising the amino acid sequence set forth in SEQ ID NO: 2 or SEQ ID NO: 4;
(c) a nucleotide sequence comprising at least 70 contiguous nucleotides of a sequence of a), or (b), wherein said sequence encodes a polypeptide having expansin activity;
(d) a nucleotide sequence having at least 60% identity with the nucleotide sequences of (a), (b), or (c), wherein said sequence encodes a polypeptide having expansin activity;
(e) a nucleotide sequence comprising a complementary sequence corresponding to the sequence of (a), (b), (c), or (d); and
(f) a nucleotide sequence that hybridizes under stringent conditions to the nucleotide sequences of (a), (b), (c), (d), or (e), wherein said sequence encodes a polypeptide having expansin activity.

8. The plant or parts thereof, of claim 7, wherein said promoter is a constitutive promoter.

9. The plant or parts thereof, of claim 7, wherein said promoter is a tissue-preferred promoter.

10. The plant or parts thereof, of claim 7, wherein said promoter is an inducible promoter.

11. The plant or parts thereof, of claim 7, wherein said promoter is a developmentally regulated promoter.

12. The plant or parts thereof, of claim 7, wherein said plant is a monocot.

13. The plant or parts thereof, of claim 12, wherein said monocot is maize, wheat, rice, barley, sorghum, or rye.

14. The plant or parts thereof, of claim 7, wherein said plant is a dicot.

15. A method for modulating brittle stalk resistance in a plant, said method comprising stably incorporating into the genome of said plant at least one nucleotide sequence operably linked to a promoter wherein said nucleotide sequence is selected from the group consisting of:
(a) a nucleotide sequence comprising the sequence set forth in SEQ ID NO: 1 or SEQ ID NO: 3;
(b) a nucleotide sequence encoding a polypeptide comprising the amino acid sequence set forth in SEQ ID NO: 2 or SEQ ID NO: 4;
(c) a nucleotide sequence comprising at least 70 contiguous nucleotides of a sequence of (a) or (b), wherein said sequence encodes a polypeptide having expansin activity;
(d) a nucleotide sequence having at least 60% identity with the nucleotide sequences of (a), (b), or (c), wherein said sequence encodes a polypeptide having expansin activity;
(e) a nucleotide sequence comprising a complementary sequence corresponding to the sequence of (a), (b), (c), or (d); and
(f) a nucleotide sequence that hybridizes under stringent conditions to the nucleotide sequences of (a), (b), (c), (d), or (e), wherein said sequence encodes a polypeptide having expansin activity.

16. A method for modulating expansin expression in a plant, said method comprising stably incorporating into the genome of said plant at least one nucleotide sequence operably linked to a promoter that drives expression in a plant cell, wherein said nucleotide sequence is selected from the group consisting of:
(a) a nucleotide sequence comprising the sequence set forth in SEQ ID NO: 1 or SEQ ID NO: 3;
(b) a nucleotide sequence encoding a polypeptide comprising the amino acid sequence set forth in SEQ ID NO: 2 or SEQ ID NO: 4;
(c) a nucleotide sequence comprising at least 70 contiguous nucleotides of a sequence of (a) or (b), wherein said sequence encodes a polypeptide having expansin activity;
(d) a nucleotide sequence having at least 60% identity with the nucleotide sequences of (a), (b) or (c), wherein said sequence encodes a polypeptide having expansin activity;
(e) a nucleotide sequence comprising a complementary sequence corresponding to the sequence of (a), (b), (c) or (d); and
(f) a nucleotide sequence that hybridizes under stringent conditions to the nucleotide sequences of (a), (b), (c), (d) or (e) wherein said sequence encodes a polypeptide having expansin activity.

17. The method of 16, wherein said promoter is a constituative promoter.

18. The method of 16, wherein said promoter is a tissue preferred promoter.

19. The method of 16, wherein said promoter is an inducible promoter.

20. The method of 16, wherein said promoter is a developmentally-regulated promoter.

21. The method of 16, wherein said plant is a dicot.

22. The method of 16, wherein said plant is a monocot.

23. The method of 22, wherein said monocot is maize, wheat, rice, barley, sorghum, or rye.

24. A method for modulating cell wall strength in a plant, said method comprising stably incorporating into the genome of said plant at least one expansin coding sequence operably linked to a Mu-tagged promoter that drives expression in a plant cell.

25. A method for identifying a recessive gene mutation in a F1 generation plant, said method comprising:
(a) crossing a Mu-active plant as a female parent with a B-A stock having a known deficient chromosome arm as a male parent;
(b) harvesting F1 seed from said female parent of said cross;
(c) planting F1 seed and allowing to grow and observing for and identifying recessive mutation phenotype in F1 plant; and
(d) identifying and selecting F1 plant demonstrating mutant phenotype of interest.

26. A method for isolating a polynucleotide encoding a wild type gene associated with the recessive mutant identified in method of claim 24, comprising utilizing said Mu-tagged recessive gene to identify and isolate said wild-type gene.

27. The method of claim 25 wherein DNA from the Mu-tagged recessive mutant F1 plant is compared to wild-type siblings for Mu element polymorphisms.

28. The method of claim 26 wherein comparison of Mu-tagged recessive mutant plant DNA to wild-type sibling plant DNA is accomplished by Southern Hybridization or PCR techniques.


SEQUENCE LISTING (9 sequences)

SEQ ID NO: 1 - DNA, 1428 bp, Zea mays
cgccgctgcccagtgcccactgctcgccgctagaggaatctgagagagagaggggaaagggcgaaggaggcgcaggcaggcatgggctccccttcctccctccccgccgcggcggcgctcgtgctcctggccctgctcgccggagcccagtgccgcgaggcccagttcgacgccgcggacgccggcgcggagaacttcaacaccagcgaggccgccgtgtactggggcccctggcagaaggcccgggccacctggtacggccagcccaacggcgccggcccggacgacaacggtggtgcgtgcggcttcaagcacaccaaccagtaccccttcatgtccatgggctcctgcggaaaccagccattgttcaaggacggcaagggatgcggctcctgctacaagattcggtgcaggaaggacccgtcctgctccgggcggacggagacggtgatcatcaccgacatgaactactacccggtgtccaagtaccacttcgacctcagcggcacggcgttcggcaggctggccaagtccggcctcaacgacaagctccgccactcgggcatcatcgacatcgagttcaccagggtgccgtgcgagttccctggcctcaagatcgggttccacgtggaggagtactcgagccccgtctacttcgcggtgctggtggagtacgaggacggcgacggcgacgtggtgcaggtggacctgatggagtccaagacggcgcgcgggccgccgacggggcgctgggcgccgatgcgcgagtcctggggctccgtctggcgcatggacaccaaccaccgcatgcagccgcccttctccatccgcatccgcaacgagtccggcaagacgctcgtcgccaggaacgtcatcccggccaactggaggcccaacaccttctaccgctccttcgtccagtacagctagctagctagctggtttgcgcccctagttcaccacccaccattattaccaccgccacccactagactactgcttctgctaccaaatactacggcggaacggaacggctggttgccgccgccgccgtcgtccttggaaaggttgaggcgtctcttggtcatccgtatcgttaccgttgtcatggtcctttgagtcgttgcaaccctgattgcaagccggcaagggggaaaaaaccaacaaagccgtgtgggaaaatggaggaggcaggcgtacaatgtacgctctcccgcccactgttgctttataatctctatatcatcatcatcttcttcttctccattccgatcggtgattaatcgaaaagtatattgtaatgtataaacaccatttcctgtttcccggagctgagctaccctgttcacacccctgtgcagggttgaggctccgcatgtttaaaaaaaaaaaaaaaaaaaaaaaaaaaaaaaaaaaaaaaaaaaaaaa

SEQ ID NO: 2 - protein, 282 aa, Zea mays
MGSPSSLPAAAALVLLALLAGAQCREAQFDAADAGAENFNTSEAAVYWGPWQKARATWYGQPNGAGPDDNGGACGFKHTNQYPFMSMGSCGNQPLFKDGKGCGSCYKIRCRKDPSCSGRTETVIITDMNYYPVSKYHFDLSGTAFGRLAKSGLNDKLRHSGIIDIEFTRVPCEFPGLKIGFHVEEYSSPVYFAVLVEYEDGDGDVVQVDLMESKTARGPPTGRWAPMRESWGSVWRMDTNHRMQPPFSIRIRNESGKTLVARNVIPANWRPNTFYRSFVQYS

SEQ ID NO: 3 - DNA, 2536 bp, Zea mays
caaccaacatggcacgttctccctcgctcctcctctctgctgctaactgcgtacacacacagacctctccctctccctcccctcccgttagctagcgggccgacgcccatctggcctataaattggcccacacggcaccacccacccaccactgctcgcactcactcactcactcacagtcgccgccgctgcccagtgcccactgctcgccgctagaggaatctgagagagagaggggaaagggcgaaggaggcgcaggcaggcatgggctccccttcctccctccccgccgcggcggcgctcgtgctcctggccctgctcgccggagcccagtgccgcgaggcccagttcgacgccgcggacgccggcgcggagaacttcaacaccagcgaggccgccgtgtactggggcccctggcagaaggcccgggccacctggtacggccagcccaacggcgccggcccggacgacaacggtgagcatgcgccgccattccgagatctctctctctctctcttaactccgtcttccccccttcttccctctccctccgtcgtgggaatgtggctcacggaacgtgtcctgtgctaggtggtgcgtgcggcttcaagcacaccaaccagtaccccttcatgtccatgggctcctgcggaaaccagccattgttcaaggacggcaagggatgcggctcctgctacaaggtacagtgaaacacagccagtatatatggatctttttttttaccagacacctctataaaaaatcagtagtatagcagtatgatctatgttttatttagcggaaacatttttttctactggttctccacttgttttagaaagaaaaaaaaatcgttgtagcaaaatttcaagagcgccataaaaaaagagattgtacactaataacaggcaggagttgtaggaccagtaactgttagagtagcacgccaaccgtacagtacagtgcaggccggtcagcacagcagctgcatgtggcccccctccgcgtgcatgtgcgcgtctctcactcgatctccgtgacagtaccatttgtatttttttttctcttaccctgcattgactcttgcttggactgacatagctgacggttgcccgcggatggcgcagattcggtgcaggaaggacccgtcctgctccgggcggacggagacggtgatcatcaccgacatgaactactacccggtgtccaagtaccacttcgacctcagcggcacggcgttcggcaggctggccaagtccggcctcaacgacaagctccgccactcgggcatcatcgacatcgagttcaccaggtcggtgcctctctttcgatttcatttgttttgtgtgttcagtttccgtaccgtatggcagcagtacagtactagtttcttcaatacctactgttgaatttggaggtagtagagagagcggaggcggcatcatctgcccaaccgctccgctcgccggacactgcatgtgcggcaaagtggcgcacctgccaaaggccacttttattgcacagcggccggcccccgaacatgctcatttttggctactagtgggcgcaaccccttttgcagcccccaacgtatctgctgcgtgcatccacggttgcaacaacgccaccggcggtgaccgaacgccagccctccttccggtttctcttgtatactaaaaaaaaaatttcgtttgcagggtgccgtgcgagttccctggcctcaagatcgggttccacgtggaggagtactcgagccccgtctacttcgcggtgctggtggagtacgaggacggcgacggcgacgtggtgcaggtggacctgatggagtccaagacggcgcgcgggccgccgacggggcgctgggcgccgatgcgcgagtcctggggctccgtctggcgcatggacaccaaccaccgcatgcagccgcccttctccatccgcatccgcaacgagtccggcaagacgctcgtcgccaggaacgtcatcccggccaactggaggcccaacaccttctaccgctccttcgtccagtacagctagctagctagctggtttgcgcccttagttcaccacccaccactactaccaccgccacccactagactactgcttctgctaccaaatactacggcggaacggaacggctggttgccgccgccgccgtcgtccttggaaaggttgaggcgtctcttggtcatccgtatcgttaccgttgtcatggtcctttgagtcgttgcaaccctgattgcaagccggcaagggggaaaaaaccaacaaagccgtgtgggaaaatggaggaggcaggcgtacaatgtacgctctcccgcccactgttgctttataatctctatatcatcatcatcttcttcttctccattccgatcggtgattaatcgaaaagtatattgtaatgtataaacaccatttcctgtttcccggagctgagctaccctgttcacacccctgtgcagggttgaggctccgcatgttaattgttctttcgtcagtaatgttttctgtgacaatgacatggaat

SEQ ID NO: 4 - protein, 282 aa, Zea mays; identical to SEQ ID NO: 2

SEQ ID NO: 5 - DNA, 27 bp, Artificial Sequence
  forward primer
accacccacccaccactgctcgcactc

SEQ ID NO: 6 - DNA, 26 bp, Artificial Sequence
  reverse primer
ccttctgcaggggccccagtacacgg

SEQ ID NO: 7 - DNA, 28 bp, Artificial Sequence
  forward primer
tgctaactgcgtacacacacagacctct

SEQ ID NO: 8 - DNA, 24 bp, Artificial Sequence
  reverse primer
tcaaccctgcacaggggtgtgaac

SEQ ID NO: 9 - DNA, 27 bp, Artificial Sequence
  TIR primer
cgccaacgcctccatttcgtcgaatcc